(19) United States
(12) Patent Application Publication
(10) Pub. No.: US 2024/0423553 A1
(43) Pub. Date: Dec. 26, 2024

(54) SYSTEMS AND METHODS FOR ESTIMATING CARDIAC EVENTS

(71) Applicant: THE UAB RESEARCH FOUNDATION, BIRMINGHAM, AL (US)

(72) Inventors: Steven G. Lloyd, Birmingham, AL (US); Thomas S. Denney, Jackson's Gap, AL (US); Muhammad Rifqi Aufan, Birmingham, AL (US)

(21) Appl. No.: 18/708,010

(22) PCT Filed: Nov. 11, 2022
(86) PCT No.: PCT/US2022/079712

§ 371 Date: May 7, 2024

Related U.S. Application Data
(60) Provisional application No. 63/278,667, filed on Nov. 12, 2021.

(51) Int. Cl. A61B 5/00; A61B 5/02; A61B 5/021; A61B 5/352; A61B 5/355

(52) U.S. Cl. CPC A61B 5/7282; A61B 5/02028; A61B 5/021; A61B 5/352; A61B 5/355; A61B 5/7239; A61B 5/7425

(57) ABSTRACT

The present disclosure describes system and methods for determining cardiac events from a physiological data signal and optionally constructing a Pressure-Volume (PV) loop display from non-simultaneously acquired measurements of pressure and volume data. One such method comprises obtaining, by a computing device, a physiological data signal of a heart of an individual; identifying, by the computing device, features of the physiological data signal and applying the features as inputs to a prediction model; determining, by the computing device using the prediction model, the cardiac events for one or more valves of the heart, wherein the cardiac events include timing of opening and closing of the one or more valves of the heart; and outputting, by the computing device, the cardiac events determined using the prediction model. The physiological data signal can be combined with non-simultaneously acquired volume data to create a PV loop display.

### CROSS-REFERENCE TO RELATED APPLICATION

This application claims priority to co-pending U.S. provisional application entitled, “Synchronization of Non-Simultaneous Pressure-Volume Data,” having Ser. No. 63/278,667, filed Nov. 12, 2021, which is entirely incorporated herein by reference.

### STATEMENT REGARDING FEDERALLY SPONSORED RESEARCH OR DEVELOPMENT

This work was supported by the U.S. Department of Veterans Affairs, and the Federal Government has certain rights in this invention.

### TECHNICAL FIELD

The present disclosure is generally related to systems and methods for measuring cardiac properties, which can include estimating cardiac events directly from physiological data signals.

### BACKGROUND

Physiological performance of the cardiac ventricles, or pumping chambers, is generally divided into a pumping function and a filling function. The performance of both the pumping and filling function can be very well described and measured by creating Pressure-Volume, or PV, loops of each heartbeat. Constructing the PV loops is highly desirable but difficult to create using practical data.

Among physiologic data, left heart catheterization (LHC) allows direct left ventricular (LV) pressure measurement and is considered to be the gold standard for measurement of for the accurate diagnosis and assessment of a number of cardiovascular diseases.[1] Since LHC is often a final, definitive test, and has a potential risk of complications, [2] gleaning all possible information from it is desirable for clinical decision making.[3,4] Currently, a number of physiologic parameters are utilized fairly commonly from LHC pressure data, including indices of systolic function and diastolic function.[5-7] Identification of aortic and mitral valve opening and closure timing is important in the assessment of left ventricle (LV) systolic and diastolic function. Knowledge of valve opening and closing can also be essential for determination of cardiac physiology, but to the inventors' knowledge, a comprehensive determination of the features of LV pressure and the association with mitral and aortic valve opening and closure has not been systematically conducted. In order to construct the PV loop using non-simultaneously acquired pressure and volume data determination of the timing of valve open and closure is needed.

### SUMMARY

Embodiments of the present disclosure describe systems and methods for determining cardiac events from a physiological data signal and optionally constructing a Pressure-Volume (PV) loop display from non-simultaneously acquired measurements of pressure and volume data. One such system comprises a processor of a computing device; and a memory in communication with the processor, in which the memory stories program instructions. Accordingly, the processor is operative with the program instructions to perform the operations of: obtaining physiological data signal of a heart of an individual; identifying features of the physiological data signal and applying the features as inputs to a prediction model; determining, using the prediction model, the cardiac events for one or more valves of the heart, wherein the cardiac events include timing of opening and closing of the one or more valves of the heart; and outputting the cardiac events determined using the prediction model. In various embodiments, an exemplary system can be configured to combine the physiological data signal with additional non-simultaneously acquired data of cardiac chamber volume, to ultimately create a PV loop.

The present disclosure can also be viewed as a method for determining cardiac events and optionally combining two types of recorded physiologic data. In this regard, one embodiment of such a method, among others, can be broadly summarized by obtaining, by a computing device, physiological data signal of a heart of an individual; identifying, by the computing device, features of the physiological data signal and applying the features as inputs to a prediction model; determining, by the computing device using the prediction model, the cardiac events for one or more valves of the heart, wherein the cardiac events include timing of opening and closing of the one or more valves of the heart; and outputting, by the computing device, the cardiac events determined using the prediction model. In various embodiments, an exemplary method can further perform synchronizing the physiological data signal by combining the physiological data signal to separately acquired volumetric data.

In one or more aspects for such systems and/or methods, the one or more valves of the heart comprise the aortic valve and the mitral valve; the physiological data signal comprises a left ventricular pressure data signal; the features comprise first and second derivatives of the left ventricular pressure data signal; the physiological data signal comprises an electrocardiogram signal; the features comprise R wave, S wave, and end of T wave features of the electrocardiogram signal; and/or the R wave, S wave, and T waves are identified by analyzing maxima and minima of the electrocardiogram signal, wherein the end of T wave is identified using a second order derivative of the electrocardiogram signal.

In one or more aspects, such system and methods further perform operations comprising synchronizing timing of the physiological data signal with another modality measurement of the heart using the timing of the opening and closing of the one or more valves of the heart; wherein the physiological data signal comprises a left ventricular pressure data signal and the another modality measurement comprises a left ventricular volume data recording of the heart that is acquired non-simultaneously with the left ventricular pressure data signal; and/or generating a synchronized pressure-volume loop display by aligning the non-simultaneously acquired left ventricular volume data with the non-simultaneously acquired left ventricular pressure data. In various embodiments, volume data can be obtained from magnetic resonance imaging (MRI), echocardiography. or other imaging modalities.

Other systems, methods, features, and advantages of the present disclosure will be or become apparent to one with skill in the art upon examination of the following drawings and detailed description. It is intended that all such additional systems, methods, features, and advantages be included within this description and be within the scope of the present disclosure.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a block diagram illustrating an exemplary computing system or device that can be utilized for systems and methods of the present disclosure.

FIG. 2 is a flow chart illustrating an exemplary method that may be implemented by computing system described with reference to FIG. 1

FIG. 3 is an Illustration of aortic and mitral valve opening and closure timing during one cardiac cycle in relation with left heart catheterization (LHC) pressure signal and its derivatives in accordance with the present disclosure.

FIG. 4 shows LHC pressure signal and its first and second derivative, where the black circles represent certain pressure signal features in accordance with embodiments of the present disclosure.

FIG. 5 shows a regression plot of cardiac timing estimates from pressure derivative features in comparison with measured timing values in accordance with the present disclosure.

FIG. 6 is a plot of the offset value between derivative features and measured timing in accordance with embodiments of the present disclosure.

FIG. 7 show plots of Bland-Altman analysis of pressure derivative features and measured timing of opening and closing of heart valves, in accordance with embodiments of the present disclosure.

FIG. 8 shows plots of heart rate dependence evaluation of pressure derivative features and measured timing offset for opening and closing of heart valves in accordance with embodiments of the present disclosure.

FIG. 9 shows an example representation of the cardiac cycle and the Pressure-Volume (PV) loop in accordance with the present disclosure.

FIG. 10 shows a flowchart describing an exemplary process for synchronizing the cardiac cycle interval difference between LV pressure data (LVP) and volume data (VOL) that are measured at different heart rates (HR).

FIG. 11 shows the adjustment of the systole and diastole interval of LV pressure data to match the total cardiac cycle interval at a volume data heart rate in accordance with embodiments of the present disclosure.

FIG. 12 shows an example systole and diastole interval adjustment of left ventricular (LV) pressure data in accordance with embodiments of the present disclosure.

FIG. 13 shows a comparison of PV loop-derived parameters between coronary artery disease and non-CAD patients that can be derived using systems and methods of the present disclosure.

FIG. 14 shows an illustration and a Doppler echocardiography (DE) image of respective aortic and mitral valve opening and closure during one cardiac cycle in accordance with the present disclosure.

FIG. 15 is a plot illustrating three main features from ECG (R, S, and T_{end}) used to estimate LV valve opening and closure timing in accordance with embodiments of the present disclosure.

FIG. 16 shows correlation plots of derived ECG features interval and DE gold standard measurement in accordance with embodiments of the present disclosure.

FIG. 17 shows plots of Bland-Altman analysis of DE and ECG-derived LV timing interval offsets on a derivative feature set in accordance with embodiments of the present disclosure.

FIG. 18 shows plots of heart rate dependence evaluation between DE and an ECG-derived LV timing interval on a derivative feature set in accordance with embodiments of the present disclosure.

FIG. 19 shows correlation plots of an ECG-derived prediction model with a DE gold standard measurement on a validation set in accordance with embodiments of the present disclosure.

FIG. 20 shows a plot of mean absolute error using percent estimation of valve timing (% RR cycle) and ECG feature-based model relative to DE measurement on a validation set in accordance with embodiments of the present disclosure.

FIG. 21 illustrates a graphical depiction of a computing system environment that can be utilized for systems and methods of the present disclosure according to one or more embodiments

### DETAILED DESCRIPTION

Disclosed herein are systems and methods for determining cardiac timing events directly from physiological data signals. As a non-limiting example, an exemplary system and/or method can utilize a computing system to perform automatic estimation of cardiac timing using neural network(s) or other machine learning or artificial intelligence methods. In one embodiment, left ventricular (LV) pressure and its time derivatives can be used to estimate valve opening and closure times, such as aortic and mitral valve opening and closures using properties of the LV pressure signal alone. Utilization of features from a pressure data signal has shown potential to allow for estimation of LV valve opening and closure timing which then can be used for the extraction of important hemodynamic information of the LV when only pressure data is available. Correspondingly, in one embodiment, LV valve opening and closure timings are estimated using electrocardiogram (ECG) imaging data. Accordingly, estimated cardiac timings can be deployed as guiding points that allow synchronization of a physiological data signal (e.g., LV pressure signals, ECG imaging data, etc.) with other modality measurements that are acquired or obtained non-simultaneously with the physiological data signal. Thus, a LV pressure signal or ECG imaging data alone can be used to estimate left-sided valve timing which can be useful in the study of cardiac systolic and diastolic physiology and pathology, and used to construct a Pressure-Volume (PV) loop (as discussed below).

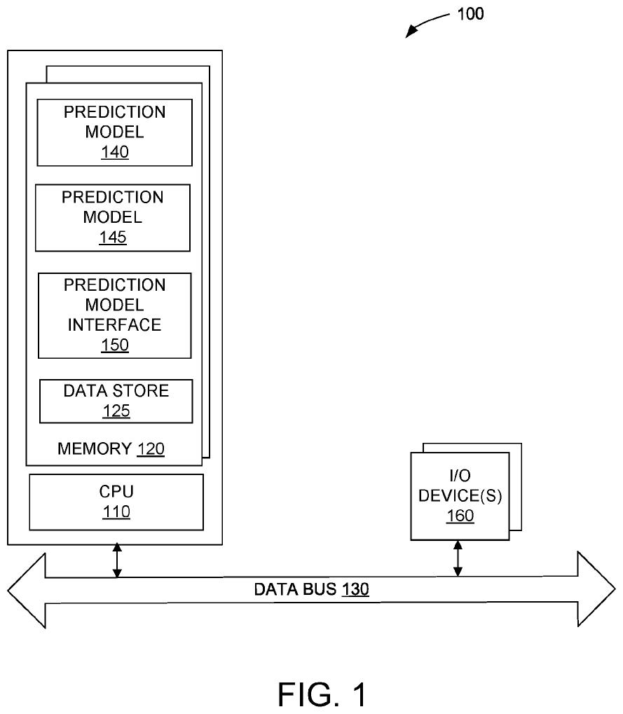

FIG. 1 is a block diagram illustrating an exemplary computing system or device 100 that can be utilized for systems and methods of the present disclosure. Computing system 100 includes at least one processor, e.g., a central processing unit (CPU), 110 coupled to memory elements 120 through a data bus 130 or other suitable circuitry. Computing system 100 stores program code within memory elements 120. Processor 110 executes the program code accessed from memory elements 120 via the data bus 130. In one aspect, computing system 100 may be implemented as a computer or other data processing system, including server computers that are accessed using browsers at client computers and/or tablets, laptops, smartphones, etc. Therefore, it should be appreciated that computing system 100 can be implemented in the form of any system including a processor and memory that is capable of performing the functions described within this disclosure.

Memory elements 120 include one or more physical memory devices such as, for example, a local memory and one or more storage devices. Local memory refers to random access memory (RAM) or other non-persistent memory device(s) generally used during actual execution of the program code. Storage device may be implemented as a hard disk drive (HDD), solid state drive (SSD), or other persistent data storage device. Computing system 100 may also include one or more cache memories (not shown) that provide temporary storage of at least some program code in order to reduce the number of times program code must be retrieved from storage device during execution.

Stored in the memory 120 are both data and several components that are executable by the processor 110. In particular, stored in the memory 120 and executable by the processor 110 are code for a prediction model of estimating cardiac timing for LV pressure data (140), code for a prediction model of estimating cardiac timing for ECG imaging data (145) and code for interfacing with the prediction model(s) and outputting a predictive outcome from the prediction model (150). Also stored in the memory 120 may be a data store 125 and other data. The data store 125 can include an electronic repository or database relevant to prediction model results. In addition, an operating system may be stored in the memory 120 and executable by the processor 110. In an embodiment, prediction model data are stored in the data store 125, such as model parameters.

For example, a prediction model may include a digitally constructed model of a probability of cardiac timing for a physiological signal, such as a LV pressure signal or ECG imaging data. In this context, the model refers to an electronic digitally stored set of executable instructions and data values, associated with one another, which are capable of receiving and responding to a programmatic or other digital call, invocation, or request for resolution based upon specified input values, to yield one or more stored output values that can serve as the basis of computer-implemented output data displays or machine control, among other things. Persons of skill in the field find it convenient to express models using mathematical equations, but that form of expression does not confine the models disclosed herein to abstract concepts; instead, each model herein has a practical application in a computer in the form of stored executable instructions and data that implement the model using the computer. The model may include a model of predicted events on the one or more fields. Model and field data may be stored in data structures in memory, rows in a database table, in flat files or spreadsheets, or other forms of stored digital data.

Input/output (I/O) devices 160 such as a keyboard, a display device, a pointing device may optionally be coupled to computing system 100, in addition to data acquisition devices such as those used to acquire physiological data signals and recordings. The I/O devices may be coupled to computing system 100 either directly or through intervening I/O controllers. A network adapter may also be coupled to computing system to enable computing system to become coupled to other systems, computer systems, remote printers, and/or remote storage devices through intervening private or public networks. Modems, cable modems, Ethernet cards, and wireless transceivers are examples of different types of network adapter that may be used with computing system 100.

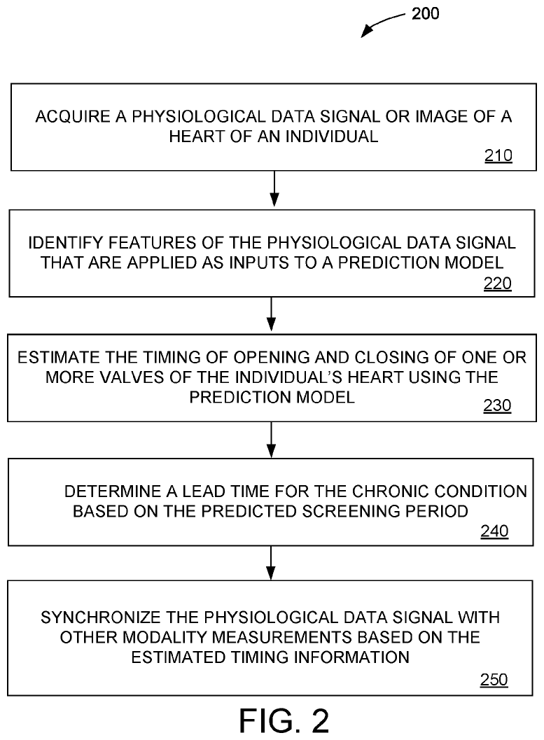

FIG. 2 is a flow chart illustrating an exemplary method 200 that may be implemented by computing system 100 described with reference to FIG. 1. Computing system 100 may execute, or include, an architecture as described generally with reference to FIG. 2. For example, the exemplary method includes modeling a probability of cardiac timing and predicting or estimating the cardiac timing of the opening and closing of valves of the heart based on a physiological data recording of the heart, such as an LV pressure signal or ECG imaging data, as discussed below. The physiological data may be obtained from anatomical measurements of the heart using ultrasound, computed tomography (CT), MRI, or other medical imaging techniques known in the art. The system 100 may obtain ECGs or electrical measurements using catheters or other sensors that measure electrical properties of the heart 120. The physiological data signals or recordings including anatomical and electrical measurements may then be stored in a non-transitory tangible media of the computing system 100. The physiological data may be transmitted to the computing device 100 from the non-transitory tangible media. Alternatively, or in addition, the physiological data may be transmitted to a server, which may be local or remote, using a network as further described herein.

In block 210, the computing system acquires or obtains a physiological data signal or image of a heart of an individual. In block 220, the computing system may identify features of the physiological data signal that are applied as inputs to a prediction model. In block 230, the prediction model of the computing system may determine or estimate the timing of opening and closing of one or more valves of the individual's heart. Thus, the computing system can output cardiac events, such as the timing of opening and closing of the one or more valves of the heart, a synchronized pressure-volume loop display, etc. Accordingly, in block 240, the computing system can synchronize the physiological data signal with other modality measurements, such as another physiological data signal of the individual's heart, based on the estimated timing information. In this way, non-simultaneously acquired physiological data recordings of the heart can be synchronized with one another.

The modality measurements may be obtained from anatomical measurements of the heart using ultrasound, computed tomography (CT), MRI, or other medical imaging techniques known in the art. The system 100 may obtain ECGs or electrical measurements using catheters or other sensors that measure electrical properties of the heart. The modality measurements including anatomical and electrical measurements may then be stored in a non-transitory tangible media of the computing system 100. The modality measurements may be transmitted to the computing device 100 from the non-transitory tangible media. Alternatively, or in addition, the physiological data may be transmitted to a server, which may be local or remote, using a network as further described herein.

Consider an exemplary embodiment of the present disclosure that involves physiological data recordings of the heart that comprise LV pressure signals. Regarding LV pressure data signals, several efforts have been made to identify the aortic and mitral valve opening and closure from the LV pressure signal directly. Several studies related with mitral valve closure (MVC) and aortic valve closure (AVC), which often describe as the end of diastole (ED) and the end of systole (ES), suggest that these timings can be estimated by the rate of change in the pressure signal. Mynard et al. described a method to identify the time of ED (and MVC) as the point of peak curvature on the pressure versus time signal just before the rapid systolic upstroke.[9] Antink et al. demonstrated the used of deconvolution of LV pressure to estimate the ED timing in sheep.[10] Abel et al. presented the use a local minimum peak of a LV pressure first derivative \dot{P}ₘᵢₙ as an indicator of end systole.[11] Mitral valve opening (MVO), on the other hand, was commonly identified by looking at the timing of left ventricle and left atrium pressure crossover.[12,13]

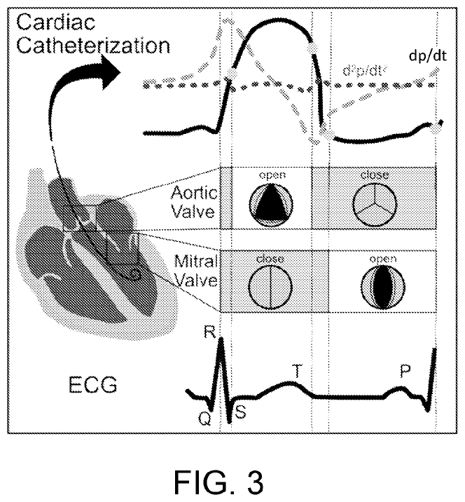

An exemplary embodiment of the present disclosure utilizes LV pressure signal features as a time marker for estimating the opening and closing of aortic and mitral valve from human left heart catheterization (LHC) data. In various embodiments, LV pressure second derivative features are utilized for automatic detection of the valve events from the pressure signal data only, as represented in FIG. 3.

To assess the merits of such systems and methods, a study was performed that included 28 participants (age 61±7 years, LVEF ≥50%) that underwent coronary angiography for chest pain and/or dyspnea evaluation for clinical indications, in which LHC included detailed LV pressure measurements. A Lead I ECG signal recorded during catheterization was included in the acquisition. Major exclusion criteria included acute myocardial infarction, coronary intervention during cardiac catheterization, atrial fibrillation, hypertrophic cardiomyopathy, myocarditis, and moderate or severe valvular disease. The study was approved by the University of Alabama at Birmingham and US Department of Veterans Affairs Institutional Review Boards. Informed consent was obtained from all patients at time of enrollment.

LV valve opening and closure timing measurement was done using Doppler echocardiography in combination with simultaneous ECG. The resulting prediction model was then used to measure LV valve opening and closure timing on ECG that simultaneously recorded with LHC pressure data and was considered to be the reference measured timing value.

After diagnostic left heart catheterization, comprehensive hemodynamic assessment was performed using a high-fidelity manometer (Millar Instruments, TX, USA or St Jude, MN, USA). Multiple LV pressure tracings were acquired. Measurements were performed for several cardiac cycles (2 to 5) and then averaged. For each participant, the first (\dot{P}) and second (\ddot{P}) time derivatives of LV pressure were calculated. Savitzkly-Golay smoothing was performed on pressure signal and its derivatives to extract key features that may be partially concealed by the noise. The pressure signal from LHC contains features that slightly vary with the wavelength; thus, the pressure signal inside that smoothing window can be approximated well with a low order polynomial. The smoothing window width (w) and polynomial order (p) to be fitted to the signal was set accordingly with ratio of w/p=5. In addition, maximum and minimum P features were used as additional references to help define the time window to consistently identify the second derivative peaks. Local minimum peaks of \ddot{P} were used to estimate the aortic valve opening (AVO) and aortic valve closure (AVC) respectively, while the first local maxima that occurred after systole was used to estimate mitral valve opening (MVO). Maximum curvature of the pressure signal kₘₐₓ around the onset of LV pressure was calculated to estimate the mitral valve closure (MVC) timing. The mathematical formula to calculate curvature k from a well-behaved curve can be expressed as:

$$k ( t ) = \frac{\ddot{P}}{{( 1 + {( \dot{P} )}^{2} )}^{3 / 2}} . \qquad Equation ( 1 )$$

The resulting offset between measured LV timing and derivative features was then calculated as measured timing—pressure derivative features.

All analysis was performed using Scipy and Statsmodel package on Python 3.8. Correlation between selected pressure derivative features and measured timing was shown using linear regression plot. Shapiro-Wilk test was performed to test normality of the data distribution. Bland-Altman plot was used to show agreement between estimated and measured value. A p-value <0.05 was considered significant.

Demographic and cardiac functional data of the 28 participants are included in Table 1. All the participants were free of arrhythmias with no evidence of acute myocardial ischemia/infarction, and had normal intervals measured. None of the subjects had bundle branch block or wide QRS. In this study group, the participant's heart rate at time of catheterization was found to be in the range of 50 to 80 bpm.

TABLE 1

| Clinical Characteristics | All (n = 28) |
|---|---|
| Age, years | 61 ± 7 |
| Sex | 25 Male, 3 Female |
| Height, cm | 176 ± 8 |
| Weight, Kg | 91 ± 17 |
| Heart Rate, bpm | 66 ± 11 |
| LVP at \dot{P}ₘₐₓ , mmHg | 58.4 ± 10.5 |
| LVP at \dot{P}ₘᵢₙ , mmHg | 76.3 ± 13.3 |
| LVP at \ddot{P}_{min 1} , mmHg | 85.0 ± 13.5 |
| LVP at \ddot{P}_{min 2} , mmHg | 108.1 ± 17.4 |
| LVP at \ddot{P}_{max 1} , mmHg | 33.2 ± 11.9 |
| LVP at k_{max,} mmHg | 22.6 ± 9.5 |

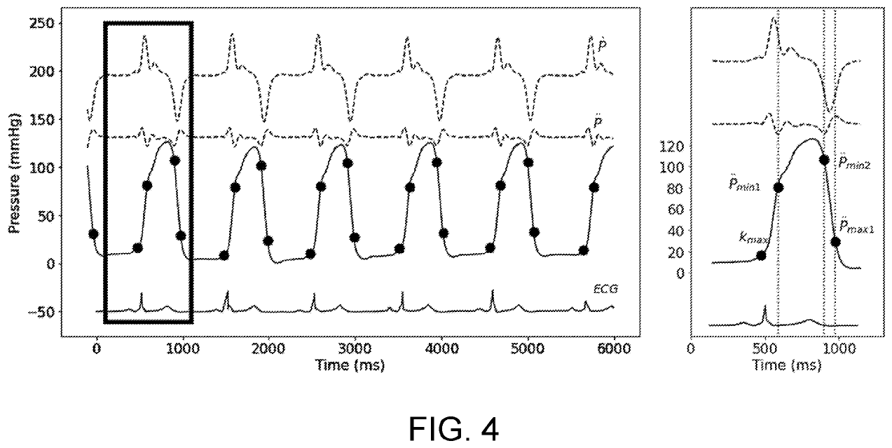

An exemplary computing system 100 performed pressure features detection on each of participant LHC pressure signal over a span of 2-5 heart beats each. \dot{P} and \ddot{P} represent the first and second derivative of the LV pressure. Three \ddot{P} features (\ddot{P}_{min 1}, \ddot{P}_{min 2}, \ddot{P}_{max 1}) and LV pressure maximum curvature during the onset of systole (kₘₐₓ) were used to estimate LV valve opening and closure timing, as represented in FIG. 4.

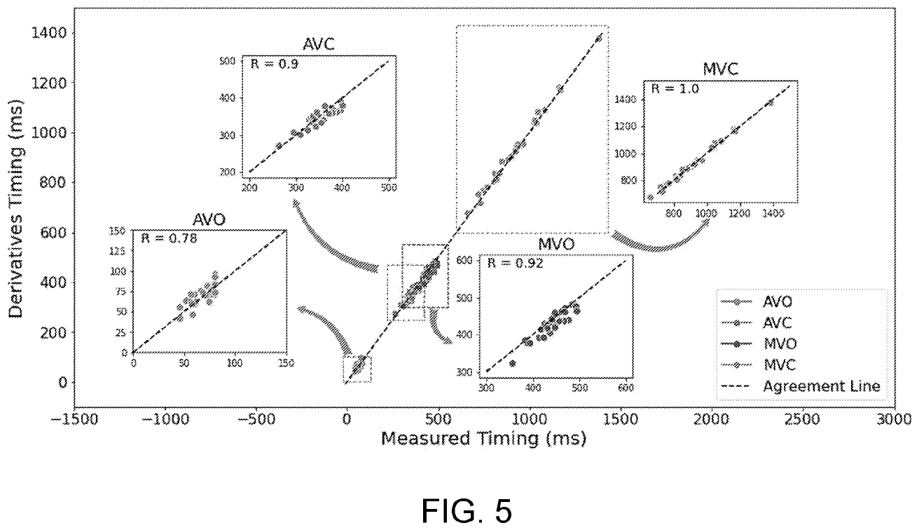

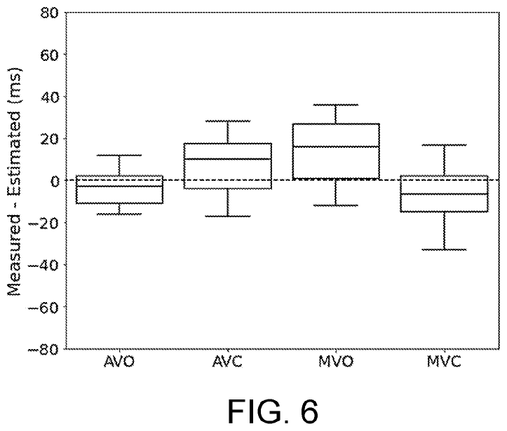

In particular, FIG. 4 shows an example of LV pressure signal and its derivatives and black circle represents estimated timing of opening and closure of the LV valve from \ddot{P} features. In one cardiac cycle, two peak features of \dot{P} and four peak features of \ddot{P} can be consistently identified. \ddot{P} peaks and kₘₐₓ were shown to be robust for quick and reliable detection and occurred relatively close to expected LV valve opening and closure time; thus, these features were selected as the main detection features. There was a strong positive correlation between the pressure derivative features and measured LV valve timing method (AVO: R=0.75, AVC: R=0.98, AVO: R=0.98, MVO: R=1.00), as shown in FIG. 5. The difference between consecutive \ddot{P} minimum peaks with measured AVO and AVC respectively was −3.5±8.4 ms and 5.8±13.5 ms. The maximum P located after the systole showed offset of 13.1±13.8 ms relative to the MVO, while kₘₐₓ showed offset of −7.1±12.9 ms relative to MVC, as shown in FIG. 6 and Table 2.

In the figure, the offset between the derivative features and measured timing was reported as means±standard deviation and displayed in box plots where the interquartile range and median compose the box and the whiskers extend from the box by 1.5× the inter-quartile range. Pressure derivative features showed low average error (<20 ms) in comparison with measured LV valves opening and closure timing. In the table, positive value describes that derivative feature occurred before the valve event, while negative value indicates the derivative feature occurred after the valve event. The results indicated a small systemic offset between the timing from HLC derivative features and measured timing.

TABLE 2

| Offset | All (n = 28) |
|---|---|
| AVO, ms | −3.5 ± 8.4 |
| AVC, ms | 5.8 ± 13.5 |
| MVO, ms | 13.1 ± 13.8 |
| MVC, ms | −7.1 ± 12.9 |

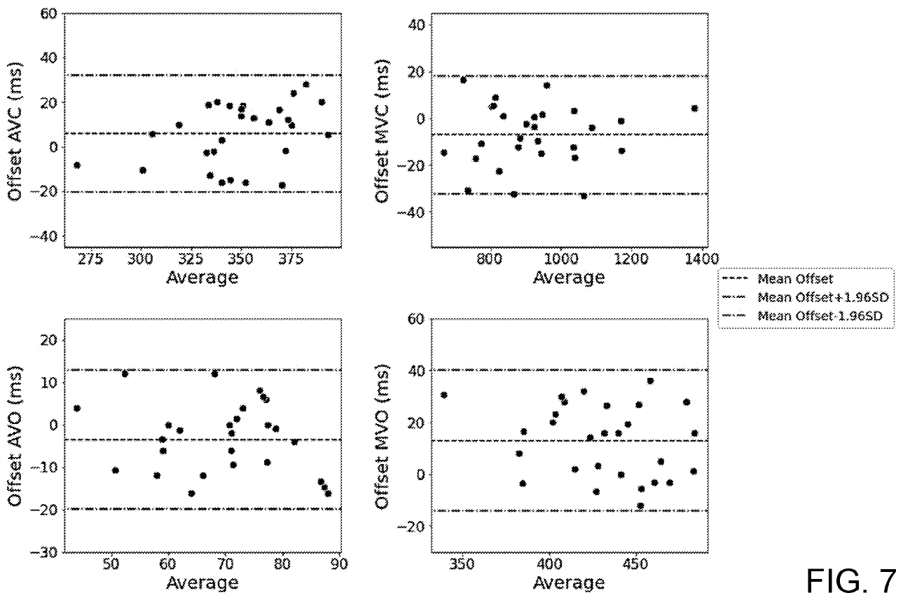

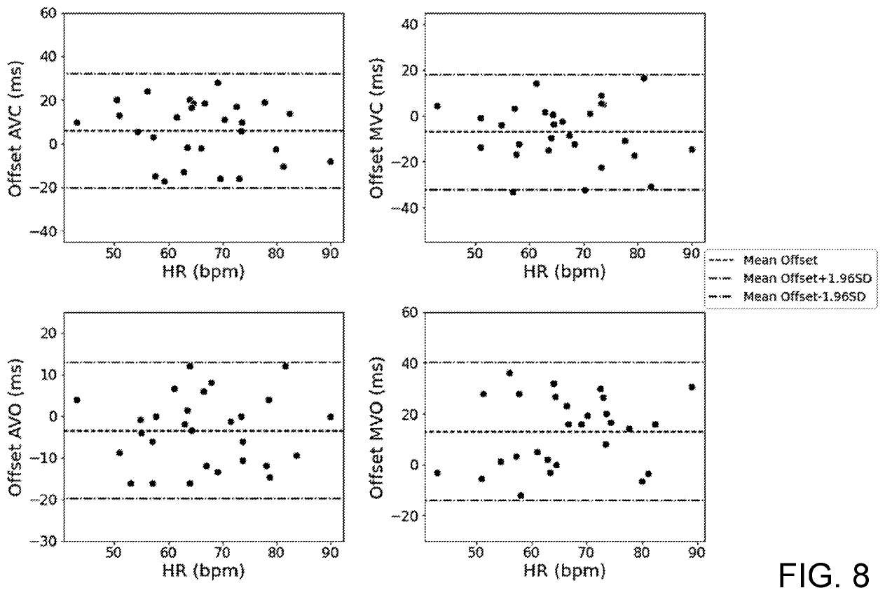

The Bland-Altman analysis was plotted in FIG. 7 which showed that there was no proportional bias in using the derivatives features to estimate measured LV valve timing and no indication of tendency toward exaggerated offset at either end of the data range. Estimated AVO and MVC from derivative features were shown to occurred slightly later, while AVC and MVO tend to occur earlier compared to measured LV valve opening and closure. Regardless, the result showed that automatic detection of LV valve timing using the LV pressure derivatives is possible without compromising the accuracy. The offset (measured timing—pressure derivative features) is also shown to be independent of heart rate, as shown in FIG. 8.

The study results indicate that P features can be identified consistently and occur very close in time to left-sided valve opening and closure. While taking the derivatives of the pressure signal might lead to noise amplification, which can lead to difficulty in identifying the maximum and minimum peak features, applying smoothing and utilizing the first derivative features as a reference to define the working window can help reduce the misdetection and make the timing measurements more reliable. In addition, the relationship between these pressure events and valve events shows no dependence on heart rate, and thus can potentially be useful as guiding points that allow synchronization of pressure data with other modality measurement that was done non-simultaneously (for example, measurement of time-dependent LV volumes).

Currently, there is no well-established reference timing marker for AVO in pressure data. While maximum P is considered to be an adequate estimation for occurrence of AVO, some studies showed that AVO tend to occur a slightly later after the maximum \dot{P}.[15-17] Since the first minimum peak \ddot{P} occurred also slightly after the maximum \dot{P}, it becomes a great candidate alternative for identifying AVO directly from an LV pressure signal.

AVC and MVC are LV valve timings that are commonly taken to occur at ES and ED. Several studies have used minimum P to identify the time of ES.[18] While the minimum \dot{P} can also be identified easily, the second minimum peak of \ddot{P} gives smaller offset relative to measured AVC time. In the case of ED, some studies have put forth effort to estimate the timing by looking at the initial onset systole region of pressure signal.[19,20] Mynard et al used the pressure “corner” of curvature to identify the ED in sheep and showed the reliability of using pressure peak curvature at the start of systolic upstroke to identify ED time.[9] In various embodiments of the present disclosure, a similar approach is used to identify the MVC and it was found that maximum curvature from LHC pressure data in human showed a similar result.

MVO is one of the LV valve timings that is relatively difficult to estimate, especially due to the gentle bend variation of the pressure signal at the beginning of diastole owing to the small drop of pressure at the end of systole until LA pressure exceeds LV pressure. While MVO can be accurately identified by simultaneously measuring LV and LA pressure and determining the time of crossover, this requires insertion of a catheter into the LA—a complex and challenging procedure that significantly increases the risk to the patient. Therefore, in the much more common situation where only LV pressure is available, resorting to the use of pressure threshold, or minimum pressure as the guiding point to define MVO might be an alternative approach. While it can work, applying an arbitrary threshold might not be reliable enough, since in some conditions, this threshold value can vary depending on the pressure signal morphology (e.g., sharpness characteristic of the bends). The second minimum peak of \ddot{P} is shown to be a good alternative candidate for identifying MVO because of its occurrence between minimum \dot{P} and minimum LV pressure (which is near the expected region of MVO), consistent and reliable detection of the feature, and relatively low offset (<20 ms) compared to the measured LV valve opening and closure timing.

Automatic detection of the LV valve opening and closure timing has a lot of potential usage in in cardiac research.[21,22] In clinical practice, identification of LV valves timing may be performed manually with the help of other external information (e.g., ECG). In addition to being time-consuming, any manual evaluation tends to be subjective and operator dependent. The utilization of pressure derivatives features can be useful as a more reliable alternative method for the automatic detection of LV valve opening and closure, free of operator bias.

In some cases, cardiac timing can be provided by obtaining an ECG signal during the image acquisition or in combination with other measurement techniques.[23-26] Although this simultaneous measurement could potentially allow for more accurate LV valves opening and closure timing estimation (e.g., ECG information can be used to extract cardiac time interval from the R-wave or T-wave), it may not always be available. The ability to estimate cardiac timing events from independent LHC pressure measurement can be important for advanced analysis of LV mechanics when simultaneous echocardiography or other means of identifying valve status and timing is not available.

Pressure-volume (PV) data provide critical information that enable quantification of important aspects of ventricular physiology parameters. In scientific cardiovascular research, the “Pressure-Volume relationship” is a central parameter that indicates the health (stiffness) of the cardiac ventricle. This requires measuring pressures (with a catheter inside the heart) and a heart chamber volume (which changes as the heart moves and squeezes) and matching the correct pressure with the correct volume. The most straightforward way to do this matching of pressure and volume data is to measure them simultaneously, but this is impractical and rarely done. Thus, since in cardiac magnetic resonance (CMR) based studies, pressure and imaging data are usually not acquired simultaneously, constructing the pressure-volume loop display can be challenging. As such, various embodiments of the present disclosure are configured to the construction of non-simultaneous pressure-volume relationships, as evidenced in the PV loop.

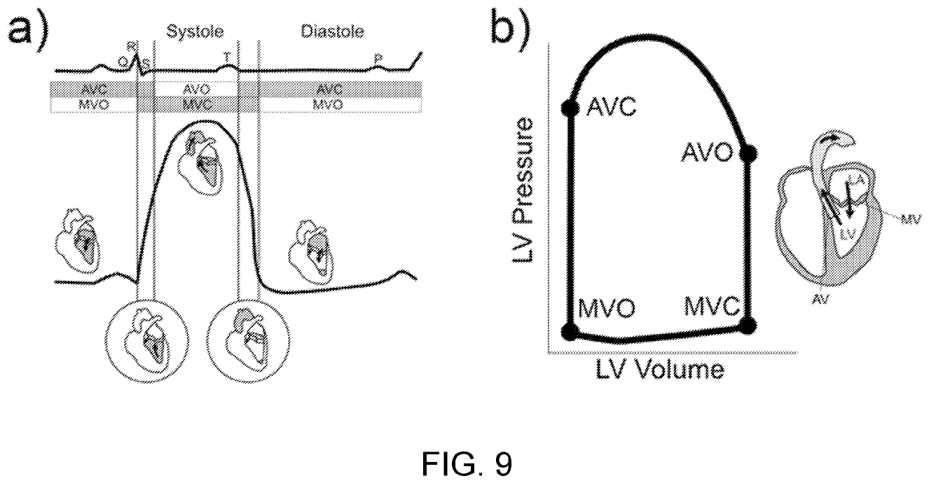

FIG. 9 shows an example representation of the cardiac cycle and the Pressure-Volume (PV) loop. At the left part (a) of the figure, the cardiac cycle is illustrated. Here, the four main phases of the cardiac cycle are shown to be (i) isovolumetric contraction (IVCT; from when mitral valve closes, MVC, to when aortic valve opens, AVO), (ii) ejection (from AVO to when aortic valve closes, AVC), (iii) isovolumetric relaxation (IVRT, from AVC to when mitral valve opens, MVO), and (iv) passive/active filling (from MVO to MVC). At the right part (b) of the figure, a PV loop display is illustrated, where the PV loop normally has a rectangular or trapezoid shape.

In general, the data available to researchers and clinicians generally consists of pressures recorded at one time with a catheter, and volumes recorded another time with either echocardiography, MRI, CT, nuclear imaging methods, etc. If heart rates are not identical—which is usually the case that they are non-identical—matching these data up with each other can be challenging. However, computer systems 100 of the present disclosure can accomplish this by synchronizing or aligning non-simultaneous measured PV data to formulate a LV PV display and demonstrate the utility of resulting PV diastolic data to measure diastolic stiffness index. The resulting PV data demonstrate an agreement with echocardiographic parameters of diastolic function assessment. This type of approach may allow for further hemodynamic parameter quantification without the need for simultaneous PV recording.

For example, in various embodiments, PV data can be synchronized by identifying aortic valve (AV) and mitral valve (MV) opening and closure timing in non-simultaneously acquired pressure and volume data. Given that the aortic valve open and close events are usually identified using echocardiography or other imaging methods, they are difficult to obtain when simultaneous measurement (echocardiography) is not available. Systems and methods of the present disclosure are able to identify features in left ventricular pressure (LVP) waveform as candidate for AV and MV opening and closures that compare to M-Mode echocardiography as the gold standard. As such, the determination of valve timings with features identified from LVP shows a strong positive correlation with M-mode measurement. Further, AV and MV timing defined using LVP waveform agrees with valve opening/closure timing determined using echo M-mode with some bias for MV timing. This finding potentially could be helpful for advanced analysis of LV mechanics when simultaneous echocardiography or other means of identifying valve status is not available.

Therefore, two different datasets (that are not acquired simultaneously) involving pressure data from inside the heart and volume data that measure the volume of the heart can be aligned based on identification of certain events in the cycle of the heart, such as the opening and closing of aortic valves. While one is able to view image data from an MRI to determine such events, these events are hard to ascertain by analyzing pressure data. Thus, in accordance with embodiments of the present disclosure, time derivatives of the pressure data have been evaluated and are determined to be predictive and indicative of certain cycle events.

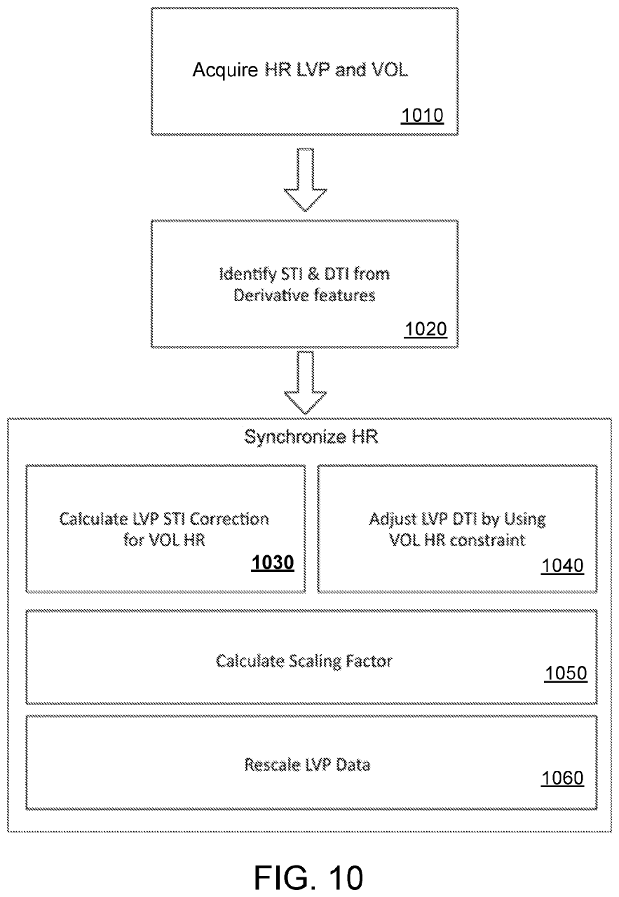

Referring back to FIG. 9, an exemplary computing system 100 of the present disclosure can synchronize pressure and volume recordings of the heart by rescaling the systolic time interval (IVCT+ejection interval) and diastolic time interval (IVRT+filling interval) from the LV pressure recordings to match the total volume data cardiac cycle interval; in general these will be different, since the data are not acquired simultaneously. Accordingly, FIG. 10 presents a flowchart describing an exemplary process for synchronizing the cardiac cycle interval difference between LV pressure data (LVP) and volume data (VOL) that are measured at different heart rates (HR). In an exemplary embodiment, the computing system 100 acquires (1010) LVP and VOL data of an individual's heart and is configured to split the pressure data into two parts and identify (1020) systolic time interval (STI) and diastolic time interval (DTI), using derivative features of the LVP. Since STI has a linear relationship with HR, the computing system 100 can correct (1030) the STI of the LVP to match the expected interval of the VOL HR and adjust (1040) the DTI with the VOL HR as a constraint accordingly. Then, the computing system 100 can calculate (1050) a scaling factor and rescale (1060) the systolic time interval and diastolic time interval from the LVP to match the VOL data.

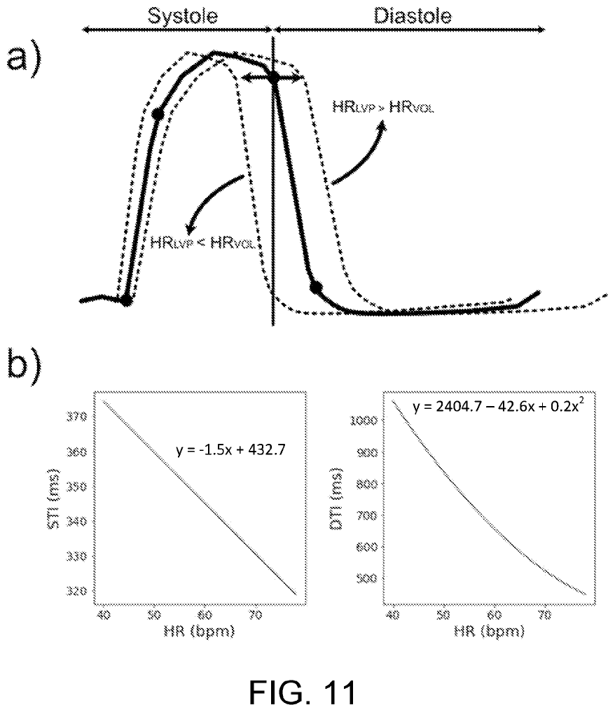

For example, the heart rate of LV pressure data may not match with the heart rate of volume data which can lead to a longer or shorter cardiac cycle in the LV pressure data. As illustrated in FIG. 11, an exemplary computing system 100 can allow the adjustment of the systole and diastole interval of the LV pressure data to match the total cardiac cycle interval at the volume data heart rate. When the heart rate of the LV pressure data (HRLVP) is lower than the volume data (HRVOL), the overall LVP cardiac cycle is going to be compressed. As a result, the higher HRLVP compared to HRVOL will lead to stretching of the LVP cardiac cycle. The relationship between STI and DTI with HR is further illustrated by the plots shown in part b of FIG. 11.

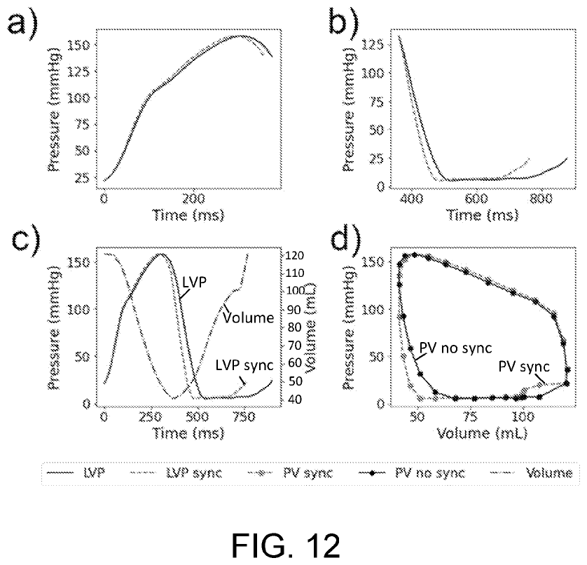

Referring now to FIG. 12, the figure shows an example systole and diastole interval adjustment of left ventricular pressure (LVP) data from heart rate 66 (LVP data) to 78 (volume data) in parts a (systole) and b (diastole), respectively. After the LVP cardiac cycle is rescaled and matched with the volume data, a synchronized Pressure-Volume (PV) loop display can then be constructed, as shown in part c of FIG. 12. A quick comparison between sync and non-sync PV loop shows that the synchronized PV data illustrates the relaxation and active filling parts of the cardiac cycle better, as illustrated in part d of FIG. 12.

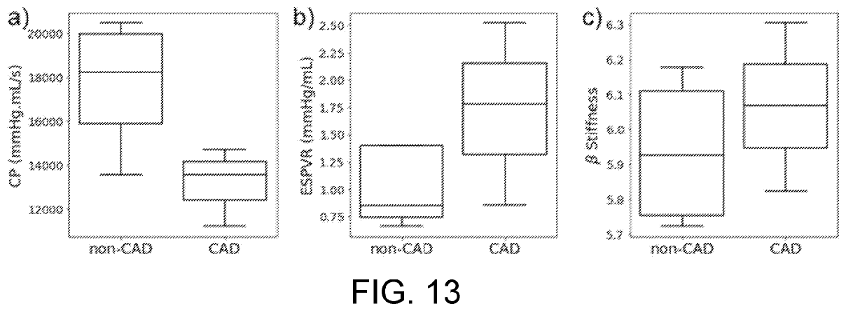

To illustrate its usefulness, FIG. 13 shows a comparison of PV loop-derived parameters between coronary artery disease (CAD; N=3) versus non-CAD (N=4) patients that can be derived using systems and methods of the present disclosure, where the center line is median value, the box represents upper and lower quartiles, and whiskers are 1.5× interquartile range. In part a of the figure, a comparison of cardiac power (CP) is provided, where cardiac power (CP) describes the efficiency and amount of work that the heart is doing (stroke work×heart rate). Part b of the figure provides a comparison of End-Systole Pressure Volume Relationship (ESPVR), while ESPVR is commonly used to describe cardiac contractility and represented as maximal pressure developed by the LV for a given volume. And, part co of the figure provides a comparison of β stiffness which is a dimensionless constant that is derived from end-diastole pressure volume relationship and can be used for indexing diastolic chamber properties.

Thus, in addition to pressure recordings, the present disclosure presents systems and methods for estimating cardiac valve timing directly from volume recordings, such as electrocardiogram (ECG) recordings. As discussed, knowledge of the timing of cardiac valve opening and closing is important in cardiac physiology. Correspondingly, the relationship between valve motion and electrocardiogram (ECG) is often assumed, however is not clearly defined. In accordance with embodiments of the present disclosure, ECG features can be used to estimate cardiac valve timings allowing useful hemodynamic information to be derived directly from ECG recordings.

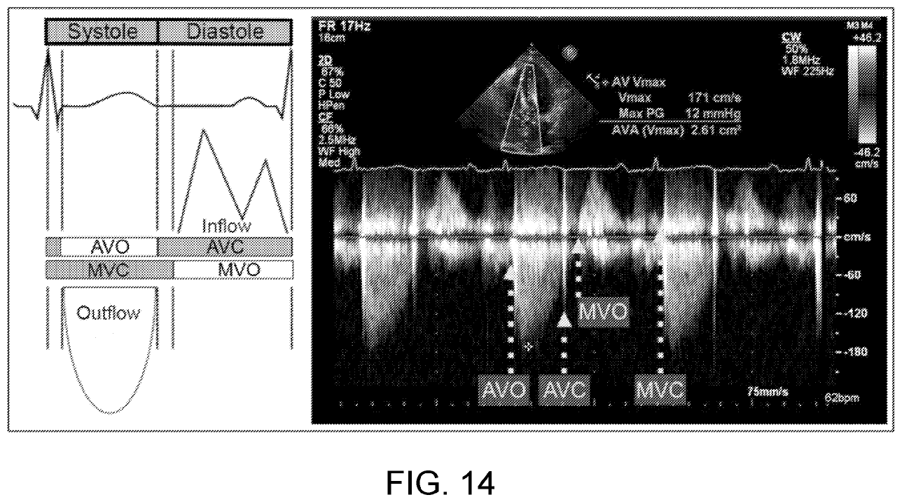

Important hemodynamic information can be obtained from the measurement of the timings of opening and closing of the cardiac valves. These valve timings are frequently used markers for evaluating electromechanical coupling of the heart, which plays an important role in heart physiology.[27] In the left ventricle (LV), the mechanical systolic time interval (ST) begins with the early isovolumic contraction followed by ejection after the aortic valve opens, as represented in the Doppler echocardiography (DE) image of FIG. 14.[28] Likewise, once the aortic valve closes, the mechanical diastolic time interval (DT) begins with isovolumic relaxation followed by LV filling after the mitral valve opens, as shown in FIG. 14.

In clinical settings, the measurement of the aortic and mitral valves opening and closure are commonly done using noninvasive assessment modalities such as Doppler flow imaging, M-mode, or tissue Doppler imaging.[29] While providing valuable information, these assessment methods can be somewhat time-consuming and require trained operators for obtaining accurate and reproducible results. Therefore, there is growing interest in the search for alternative approaches to measure the cardiac intervals.[25-26, 30-32] Techniques that allow quick and accurate estimation of these cardiac events would be particularly useful.

Even with the vast and growing array of imaging and other diagnostic technologies for evaluation of patients with cardiovascular disease, the electrocardiogram (ECG) maintains a central role, due to its usefulness, its simplicity, and its excellent benefit-cost relationship, across a wide variety of disease conditions. [33-35] With the ECG providing evidence of onset of depolarization and repolarization of the atria and ventricles, it is a key marker of electrical systole and diastole, but the relationship to mechanical action (electro-mechanical coupling), at least in terms of valve opening and closure timing, has only been evaluated in limited circumstances. Here, the present disclosure determines the specific relationships between ECG signal features and timing of aortic and mitral valve opening and closing, compared to the ‘gold standard’ Doppler echocardiography (DE) flow imaging measurement. Such an approach, performed by systems and methods of the present disclosure, can be useful for a number of applications, including synchronizing physiologic data acquired under different heart rate conditions.

To assess the merits of such systems and methods, a study was performed that included 37 patients (age 64±8 years, LV ejection fraction ≥50%, 62% males) that came from a group of patients that underwent echocardiography for chest pain and/or dyspnea evaluation for clinical indications. Diagnostic testing for their symptoms revealed that a subset of the patients had coronary artery disease (CAD, defined as obstruction of major coronary arteries >50% and/or prior history of coronary revascularization, n=12), chronic obstructive pulmonary disease (COPD) with mild to moderate airflow obstruction (n=10), or combination of both (n=6). All patients were in sinus rhythm at the time of the study. The dataset was initially randomly sorted according to presence or absence of CAD and COPD, and then divided by odd-even split into derivative set (19 patients) and validation set (18 patients). In the derivation set, there were 6 patients with CAD, 5 with COPD, and 3 patients had both CAD and COPD. In the validation set, there were 6 patients with CAD, 5 with COPD, and 3 with both CAD and COPD. The study was approved by the University of Alabama at Birmingham and US Department of Veterans Affairs Institutional Review Boards. Informed consent was obtained from all the patients at time of enrollment.

A standard 12 Lead ECG was recorded on the day of the echocardiogram, and digital calipers were used to measure heart rate and standard intervals (QRS width, raw and corrected QT intervals using the Bazett formula, PR interval). Echocardiographic study was performed at rest in supine position in all standard views by a trained operator using an iE33 system equipped with S5-1 probe (Philips Medical System, Andover, MA, USA). All included patients had normal LV ejection fraction and LV size, assessed by an expert echocardiographer according to ASE recommendations.[36] Continuous wave and pulse wave DE in the apical four or five-chamber view were used to measure valve timing in relation to the R wave from a Lead I ECG (left arm potential-right arm potential) that was recorded simultaneously on the DE image, as shown in FIG. 14.

The annotations of the aortic valve opening (AVO), aortic valve closure (AVC), mitral valve opening (MVO), and mitral valve closure (MVC) from DE images were performed by trained operators (Z.T.J, N.J.M., S.G.L). The AVO was annotated as the onset of the ejection of the left ventricle as seen on DE of the LV outflow tract, while the AVC was defined immediately at the end of the flow. MVO and MVC were annotated at the onset of the E wave and the end of the A wave. The opening and closure timing were then measured using ECG R wave as the reference (zero time point). Measurements were performed for several cardiac cycles (2 to 4) recorded in DE imaging and then averaged.

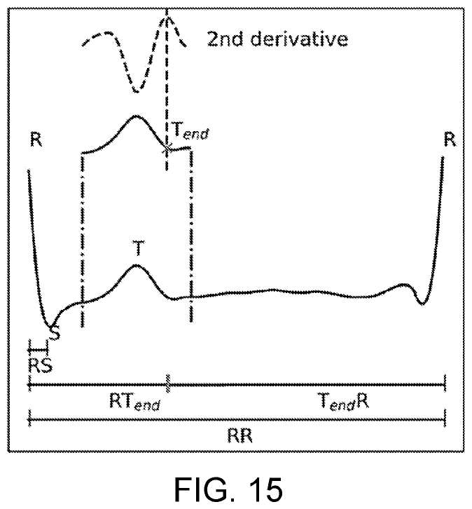

A Lead I ECG signal recorded during DE was included on the DICOM image display of the DE acquisition. Screen captures of the ECG in the image were isolated and manually processed using WebPlotDigitizer version 4.5 (Automeris, Pacifica, CA USA; https://automeris.io/WebPlotDigitizer) to obtain the time series data. The recorded data were then preprocessed using high pass and low pass filters to reduce baseline wander and noise. ECG recordings were acquired at a sampling rate of 250 Hz. From the preprocessed ECG signal, the following features are evaluated: amplitude, morphology and duration of its waves, intervals and segments as well as their appearance sequence. R, S, and T waves were identified by analyzing the local maxima and minima of the ECG signal. End of T wave was identified by using derivative features of the ECG: additional smoothing using Savitzki-Golay filter was applied, followed by the calculation of the 2nd-order derivative of the signal. Search window that was positioned after the T wave was set to identify peak that represented the ‘shoulder’ of T wave which was used as the feature for identifying end of T wave, as represented in FIG. 15. P wave timing was not included due to its low signal-to-noise ratio (SNR) and variability, causing reliable identification of the peak from the ECG signal difficult. Once these features from the ECG were identified, a data model was applied to determine the relationship between the identified ECG feature and the valve opening and closing as determined by DE. Individualized linear models as a function of the ECG feature timing were chosen as follows:

$$A V O = S + {Δ}_{1} \qquad Equation 2$$

$$A V C = {T}_{end} + {Δ}_{2} \qquad Equation 3$$

$$M V O = {T}_{end} + {Δ}_{3} \qquad Equation 4$$

$$M V C = R + {Δ}_{4} \qquad Equation 5$$

where Δ represents offset that was fitted from average value (among the 19 patients from derivative set) of the difference between the ECG features (S, T_{end}, R) and measurement from DE.

Using the models based on ECG features, the estimation of aortic and mitral valve opening and closure timing was compared with a reference model based only on cardiac timing estimation using percentages of the RR interval, to determine if the RR interval alone (i.e., inverse of the heart rate) is sufficiently accurate in estimating valve opening and closure using the validation set. All analysis was performed using Scipy, Statsmodel, and Scikit-Learn package on Python 3.8. Linear regression was performed to show the association between the timings of valve opening and closure directly measured from DE images and from ECG-features derived model. Bland-Altman method was used to assess data variability and bias. Shapiro-Wilk test was performed to test normality of the data distribution. Two-way analysis of variance (ANOVA) was used to assess how CAD, COPD, or interaction of these two conditions affect offset LV valves timing measurement. Comparison of mean absolute error (MAE) between the methods using percent change of RR interval versus ECG features with correction was performed using Mann-Whitney U test. A p-value <0.05 was considered significant.

Demographic and cardiac functional data of the 37 patients are included in Table 3. On the 12 lead ECG, all the patients were free of arrhythmias with no evidence of acute myocardial ischemia/infarction, and had normal intervals measured, except for one participant with a mildly prolonged QTc of 500 ms, as shown in Table 4. None of the subjects had bundle branch block or wide QRS.

TABLE 3

| Clinical Characteristics | All (n = 37) |
|---|---|
| Age, years | 64 ± 8 |
| Sex | 23 Male, 14 Female |
| Height, cm | 173 ± 8 |
| Weight, kg | 85 ± 18 |
| Heart Rate, bpm | 68 ± 14 |

TABLE 4

| Parameter | All (n = 37) |
|---|---|
| Heart Rate, bpm | 72 ± 18 |
| RR, ms | 867 ± 185 |
| PR, ms | 160 ± 21 |
| QRS, ms | 87 ± 13 |
| QT, ms | 396 ± 45 |
| QTc, ms | 421 ± 31 |

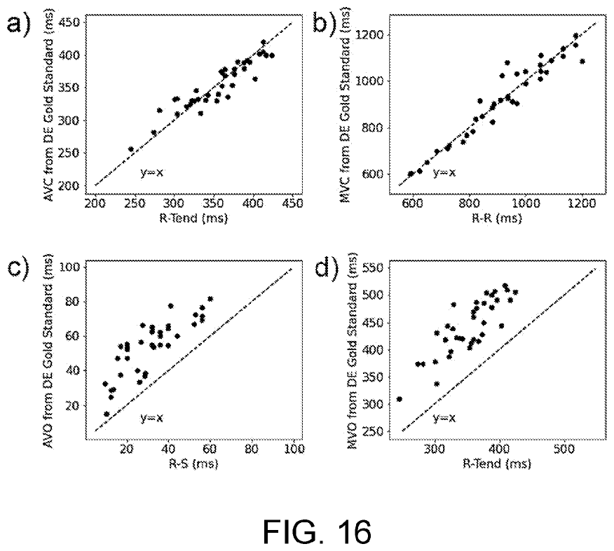

In this study group, the participant's heart rate at time of DE was found to be in the range of 50 to 90 bpm. Opening and closure timing of the LV valves measured from the R wave using DE is summarized in Table 5. Average timing of ECG features that were used to estimate LV valves opening and closure timing (R, S, T_{end}) from DE is summarized in Table 6. Compared to standard ECG analysis, S timing is similar but not identical to QRS width, whereas T_{end} is similar to QT interval. A two-way ANOVA was performed to analyze the effect of CAD and COPD on the timing offset between ECG features and DE measurement, and it was found that there was no statistically significant effect of CAD (p=0.70), COPD (p=0.21), or interaction between these two conditions (p=0.97) on the timing offset for any of the AVO, AVC, MVO, or MVC. The correlation plots of the ECG features and the DE gold standard measurement are shown in FIG. 16. Here, it was observed that AVC and MVC were well coincident with the timing of T_{end} and R, while AVO and MVO had a fixed timing offset relative to DE gold standard measurement.

TABLE 5

| Valve Event | All (n = 37) |
|---|---|
| AVO | 54 ± 16 |
| AVC | 353 ± 37 |
| MVO | 443 ± 52 |
| MVC | 916 ± 169 |

TABLE 6

| Parameter | All (n = 37) |
|---|---|
| S | 31 ± 14 |
| T_{end} | 354 ± 43 |
| RR (to next beat) | 914 ± 169 |

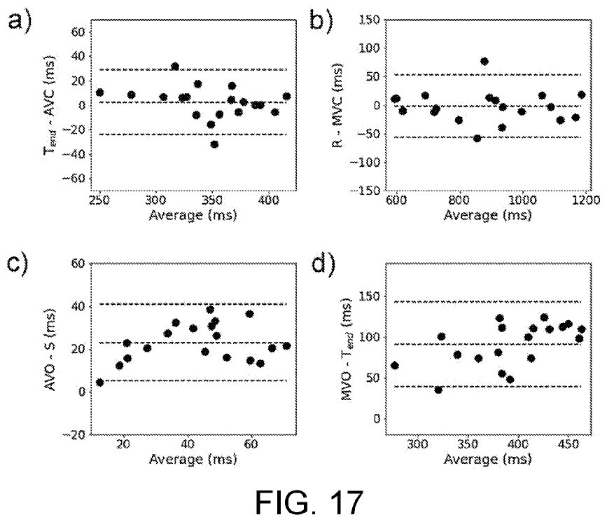

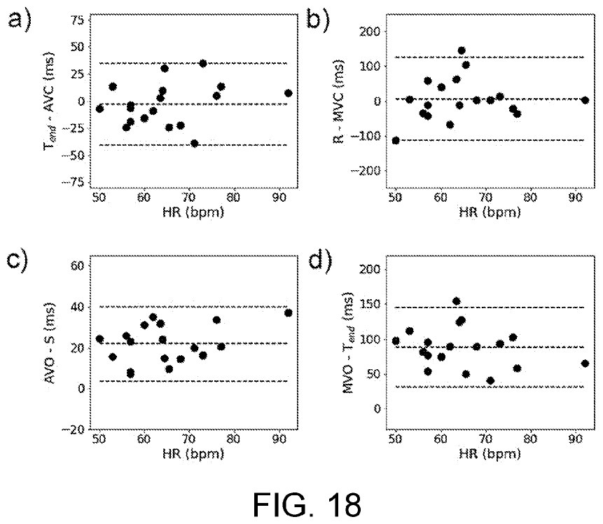

The derivative set was used to obtain the offset correction. T_{end} and R wave were used as ECG features to identify AVC and MVC, respectively, and were compared to AVC and MVC timing measured by DE. Accordingly, T_{end} and R wave showed excellent agreement with AVC and MVC measurement using DE with offsets of 2±13 ms and 2±27 ms (as shown in FIG. 17 at parts a and b), indicating that on average, the AVC and MVC occurred very close to the T_{end} and R wave, with Δ₂ and Δ₄ therefore approximately zero from Equations 3 and 5. For identification of AVO and MVO, S wave and T_{end} were used as index points, since there were no identifiable ECG features in the immediate temporal vicinity of these valve events. AVO occurred 23±9 ms after the S wave (as shown in FIG. 17 at part c); defined now as Δ₁ from Equation 2, while MVO occurred 90±26 ms after the T_{end} (as shown in FIG. 17 at part d); defined as Δ₃ in Equation 4. All of these offsets were also found to be independent of heart rate, as shown in FIG. 18.

The use of the R wave to determine AVO was also examined, since it was a prominent feature that was easy to identify on the ECG. However, the use of the R timing to identify AVO was found to cause proportional bias with the variation of heart rate when compared to AVO on the DE measurement (data not shown). Hence, R wave was not used to estimate AVO.

Using the measured offsets (as shown in Table 7), a linear prediction model was built to estimate the LV valve opening and closure timing as described in Equations 2-5. AVC and MVC timing was estimated by adding the average offset from each measurement to the T_{end} and R wave, respectively, as follows: AVC=T_{end}+ (2±13) ms and MVC=R+ (−2±27) ms. Applying the same approach for AVO and MVO by using S and T_{end} as the reference point, the linear prediction model estimated AVO and MVO timing events as follows: AVO=S+ (23±9) ms and MVO=Tend+(90±26) ms.

TABLE 7

| Offset | Derivative set (n = 19) |
|---|---|
| Δ₁, ms | 22 ± 9 |
| Δ₂, ms | 2 ± 13 |
| Δ₃, ms | 90 ± 26 |
| Δ₄, ms | −2 ± 27 |

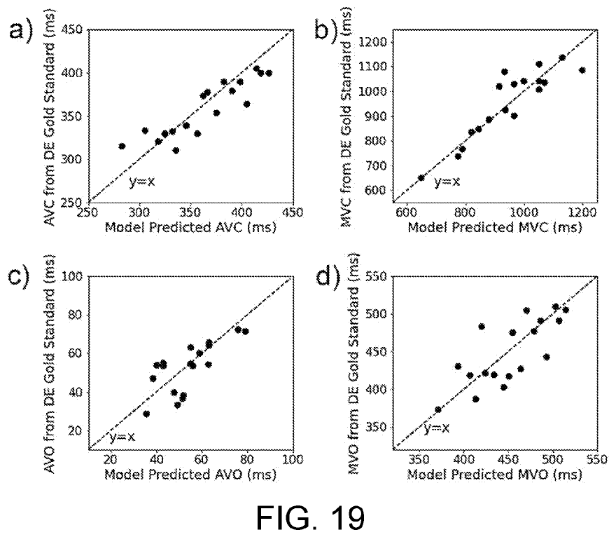

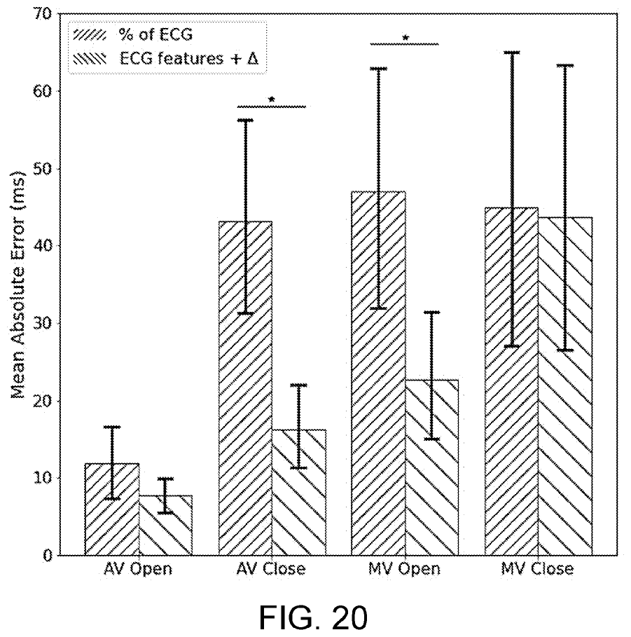

The performance of the linear models was evaluated using the validation set that was not used in previous steps. The agreement of the prediction model and DE gold standard measurement is shown in FIG. 19, where the dashed line depicts absolute agreement. The mean absolute error (MAE) of the model with respect to the validation set for AVO, AVC, MVO, and MVC were 8±5 ms, 16±11 ms, 23±18 ms, 44±42 ms, respectively, as shown in FIG. 20.

Next, the estimation of LV valves opening and closure timing using an exemplary prediction model based on ECG features (as described above) was compared with a reference model based only on cardiac timing estimation using percentages of the RR interval. While we are not aware of the reference model being used specifically to estimate timing of valve opening and closings, it is widely used in applications such as cardiac CT imaging, where specific gating of the image to time periods in the cardiac cycle is desired (e.g., mid-diastole with minimal motion of the left coronary artery branches occurring at approximately 70% of the RR cycle; end-systole at near 30% of the RR cycle). Thus, using the reference approach as a point of comparison, again using the DE as the gold standard, the reference timings were AVO=6%±2% RR, AVC=41%±6% RR, MVO=51%±7% RR, MVC=100%±1% RR. MAE calculated from the validation set using our ECG feature-based model with the reference RR % models were compared, as shown in FIG. 20, and except for the AVO and MVC, the exemplary prediction model based on ECG features yielded a significantly better accuracy (lower MAE) than the % RR interval reference approach. This is perhaps not surprising, since the exemplary prediction model using the ECG features incorporates additional information allowing potential factors influencing electrical myocardial activation and subsequent effects on mechanical ventricular and atrial activation.

As such, the foregoing study explores the use of ECG features to automatically identify the LV valve opening and closuring timing from the ECG signal data only. In clinical cardiology, an ECG is usually the first test to be employed in evaluating cardiac function. The ECG tracks and amplifies the changes in electrical potential due to cardiac depolarization and repolarization for each heartbeat and provides a wealth of information about heart rhythm, as well as cardiac morphology and function.

ECG has been the core tool for diagnosis and management of cardiovascular diseases. Recently, there has been interest in using ECG as to automatically detect and classify cardiac abnormalities, including a study by Kashou et al. [37] where ECG features were used to identify preclinical LV systolic dysfunction and a study by Vaid et al. where they demonstrated the use of ECG for inexpensive screening and diagnostic tools to evaluate ventricular functions. [38] Recently, a study of Schlesinger et al. highlights the important insight that can be extracted from ECG by developing a novel deep learning model from 12-lead ECG data to identify elevated mean pulmonary capillary wedge pressure.[39] When evaluating cardiac systolic and diastolic function, knowledge of valve opening and closing to determine ejection and filling times as well as isovolumic periods, can be important.

Therefore, the present disclosure examined the timing of aortic and mitral valve opening and closure from the ECG using modern digital signal processing methods. To the inventors' knowledge, the relationship between the features of the ECG and the opening and closing of the aortic and mitral valves has not been previously systematically evaluated. Perhaps, the relationship most studied has been that between the ECG and the aortic valve closure. Several studies have tried to investigate the relationships between ECG and the timing of aortic valve closure.[40, 41] Using seismocardiography for determining aortic valve closure, it was reported that the aortic valve closure occurs at the end of T wave in young healthy subjects at rest.[23] It was also found that an increase in heart rate with atrial pacing did not change the timing of aortic valve closure compared to the end of T wave, whereas beta-adrenergic stimulation with isoproterenol administration did affect the relationship between the end of the T wave and valve closure.[24] It is important to note that a number of different techniques have been used to identify end of T wave: intersection of lines, threshold on the amplitude of T wave, threshold on the first derivative of ECG signal, trapezium-based approach etc.[42, 43] The peak second derivative is commonly used in various peak analyzer algorithms. Since taking the second derivative can amplify the signal in the original data, the second derivative can be used to detect hidden features in data. Changes in the second derivative values indicate changes in the ECG shape (characteristic of the ECG T wave ‘shoulder’ signal). In the present disclosure, T_{end} was found, by the digitally processed second derivative, to agree well with the timing of AVC, with a very small offset (2±13 ms)—thus, the AVC occurs at T_{end} (representing the end of repolarization of the ventricles) to a very high degree of accuracy.

The R wave is commonly used to estimate timing of end diastole because it is readily available, and the R-wave is generally the most easily detected feature of the ECG. The present disclosure shows that in the study population, even though the ECG R-wave sometimes occurs before the onset of mitral valve closure, it tends to normally occur after. Several studies have examined the mechanism of MVC and its timing of occurrence, and the classic view was that the MVC occurred as the result of systolic onset with ventricular contraction which led to an increase in LV pressure that overcomes the left atrial pressure, causing the mitral valve to close. In this study, MVC was found to actually occur 2±26 ms before the R wave. While increase in LV pressure due to LV mechanical contraction (which would necessarily occur after the R wave) can certainly close the mitral valve, the mechanism of MVC is complex, and could depend on loading conditions and other factors. One such possible example considers that MVC may occur at the end of atrial contraction and beginning of atrial relaxation, producing a drop in pressure in the left atrium near the area of the valve cusps, with blood inflow into this area from the endocardial regions of the LV causing the valve leaflets to closed before the start of ventricular contraction.[44] The normal MVC could occurred as the result of combination of these two mechanisms. In the ECG MVC model, the complex regulation of MVC is likely a main contributor to the lower ability of the model to predict MVC using the ECG alone, as evidenced by the fact that among the exemplary prediction models for AVO, AVC, MVO, and MVC, MVC had the largest mean absolute error among other ECG valve timing models, when compared to the DE gold standard.

While there are no prominent ECG features in the immediate vicinity of AVO and MVO, the present disclosure utilizes a relationship with the S and T waves, respectively, which allowed good prediction of these events. Accordingly, the AVO can be identified with the S wave indicating final depolarization of the LV, as pressure in the LV increases during isovolumetric contraction, eventually exceeding the pressure in the aorta. The nearest identifiable feature in the ECG to the MVO is the T wave, allowing this portion of the ECG signal to estimate MVO, such that MVO will occur after closure of the aortic valve (with the difference designated as the isovolumic relaxation time, or IVRT). It is recognized that variability in IVRT, which is known to be influenced by abnormalities in active diastolic relaxation, could complicate this approach and make it less predictive of true MVO in a general population. To further examine this, we explored the possibility of influence of COPD and CAD, which is known to influence diastolic relaxation and the IVRT on the validity of the ECG prediction model to predict MVO.[45, 46] It was found that the exemplary ECG model worked equally well, with no significant difference in the offset timings regardless of presence or absence of CAD, COPD, or interaction of these two conditions.

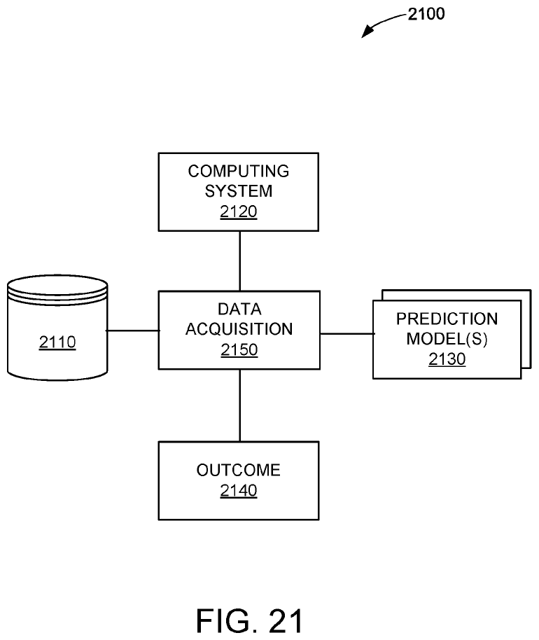

FIG. 21 illustrates a graphical depiction of a computing system environment 2100 that can be utilized for systems and methods of the present disclosure according to one or more non-limiting embodiments. The computing system environment 2100 includes data 2110 (e.g., physiological data signal, signal features, etc.), a computing system 2120 (e.g., computing system 100), one or more prediction models 2130, a plurality of outcomes 2140 (e.g., cardiac timing estimates), and underlying data acquisition hardware 2150 (e.g., electrodes, ECG monitor, LV pressure instrumentation, etc.).

In general, the computing system environment 2100 uses at least a portion of the data 2110 to train the computing system 2120 while building the prediction model 2130 to enable the plurality of outcomes 2140 to be predicted. In such a configuration, the computing system environment 2100 may operate with respect to the data acquisition hardware 2150 to train the computing system 2120, build the prediction model 2130, and predict outcomes using one or more algorithms. These algorithms may be used to solve the trained model 2130 and predict outcomes 2140 associated with the data acquisition hardware 2150.

Computer program code for carrying out operations of the present disclosure may be written in a variety of computer programming languages and stored in non-transitory computer readable media. The program code may be executed entirely on at least one computing device (or processor), as a stand-alone software package, or it may be executed partly on one computing device and partly on a remote computer. In the latter scenario, the remote computer may be connected directly to the one computing device via a LAN or a WAN (for example, Intranet), or the connection may be made indirectly through an external computer.

In the context of this document, a “computer-readable medium” can be any means that can contain, store, communicate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium. More specific examples (a nonexhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM or Flash memory) (electronic), an optical fiber (optical), and a portable compact disc read-only memory (CDROM) (optical). In addition, the scope of the certain embodiments of the present disclosure includes embodying the functionality of the various embodiments of the present disclosure in logic embodied in hardware or software-configured mediums.

It should be emphasized that the above-described embodiments of the present disclosure are merely possible examples of implementations, merely set forth for a clear understanding of the principles of the disclosure. Many variations and modifications may be made to the above-described embodiment(s) without departing substantially from the principles of the present disclosure. All such modifications and variations are intended to be included herein within the scope of this disclosure.

### REFERENCES

1. Nishimura R A, Carabello B A. Hemodynamics in the Cardiac Catheterization Laboratory of the 21st Century. Circulation 2012; 125:2138-2150. doi:10.1161/CIRCULATIONAHA. 111.060319.
2. Manda Y R, Baradhi K M. Cardiac Catheterization Risks and Complications. StatPearls, Treasure Island (FL): StatPearls Publishing; 2022.
3. Abbas A E, Pibarot P. Hemodynamic characterization of aortic stenosis states. Catheterization and Cardiovascular Interventions 2019; 93:1002-1023. doi:10.1002/ccd.28146.
4. Yannopoulos D, Bartos J A, Aufderheide T P, Callaway C W, Deo R, Garcia S, et al. The Evolving Role of the Cardiac Catheterization Laboratory in the Management of Patients With Out-of-Hospital Cardiac Arrest: A Scientific Statement From the American Heart Association. Circulation 2019; 139:e530-e552. doi:10.1161/CIR.0000000000000630.
5. Kern M J, Christopher T. Hemodynamic rounds series II: The LVEDP. Catheterization and Diagnosis Cardiovascular 1998; 44:70-74. doi:10.1002/(SICI) 1097-0304 (199805) 44:1<70::AID-CCD17>3.0.CO;2-T.
6. Moscucci M. Grossman & Baim's cardiac catheterization, angiography, and intervention: Eighth edition. 2013.
7. Morton J. Kern P S. Cardiac Catheterization Handbook (6th Edition). n.d.
8. Voorhees A P, Han H-C. Biomechanics of Cardiac Function. Compr Physiol 2015; 5:1623-1644. doi:10.1002/cphy.c140070.
9. Mynard J P, Penny D J, Smolich J J. Accurate automatic detection of end-diastole from left ventricular pressure using peak curvature. IEEE Trans Biomed Eng 2008; 55:2651-2657. doi:10.1109/TBME.2008.2001295.
10. Antink C H, Ruschen D, Leonhardt S, Walter M. Estimation of end-diastolic pressure via deconvolution. 2016 Computing in Cardiology Conference (CinC), 2016, p. 185-188.
11. Abel F L. Maximal negative dP/dt as an indicator of end of systole. Am J Physiol 1981; 240: H676-679. doi:10.1152/ajpheart. 1981.240.4.H676.
12. Buckberg G D, Nanda N C, Nguyen C, Kocica M J. What Is the Heart? Anatomy, Function, Pathophysiology, and Misconceptions. J Cardiovasc Dev Dis 2018; 5:33. doi:10.3390/jcdd5020033.
13. Tsakiris A G, Gordon D A, Padiyar R, Frechette D. Relation of mitral valve opening and closure to left atrial and ventricular pressures in the intact dog. American Journal of Physiology-Heart and Circulatory Physiology 1978; 234:H146-H151. doi:10.1152/ajpheart. 1978.234.2.H146.
14. Balmer J, Smith R, Pretty C G, Desaive T, Shaw G M, Chase J G. Accurate end systole detection in dicrotic notch-less arterial pressure waveforms. J Clin Monit Comput 2021; 35:79-88. doi:10.1007/s10877-020-00473-3.
15. Adler D, Nikolic S D, Sonnenblick E H, Yellin E L. Time to dP/dtmax, a preload-independent index of contractility: open-chest dog study. Basic Res Cardiol 1996; 91:94-100. doi:10.1007/BF00788870.
16. Mason D T. Usefulness and limitations of the rate of rise of intraventricular pressure (dp/dt) in the evaluation of myocardial contractility in man*. The American Journal of Cardiology 1969; 23:516-527. doi:10.1016/0002-9149 (69) 90005-8.
17. Wildenthal K, Mierzwiak D, Mitchell J. Effect of sudden changes in aortic pressure on left ventricular dp/dt. American Journal of Physiology-Legacy Content 1969; 216:185-190. doi:10.1152/ajplegacy.1969.216.1.185.
18. Alyono D, Larson V E, Anderson R W. Defining end systole for end-systolic pressure-volume ratio. J Surg Res 1985; 39:344-350. doi:10.1016/0022-4804 (85) 90113-1.
19. Kass D A, Midei M, Brinker J, Maughan W L. Influence of coronary occlusion during PTCA on end-systolic and end-diastolic pressure-volume relations in humans. Circulation 1990; 81:447-460. doi:10.1161/01.CIR.81.2.447.
20. Rynning S E, Brunvand H, Birkeland S, Hexeberg E, Grong K. High afterload during 10 min of regional ischaemia affects diastolic creep but not systolic function in reperfused (stunned) myocardium. Acta Physiologica Scandinavica 1996; 158:39-44. doi:10.1046/j. 1365-201X.1996.520295000.x.
21. Wajdan A, Jahren T S, Villegas-Martinez M, Khan F H, Halvorsen P S, Odland H H, et al. Automatic Detection of Aortic Valve Events Using Deep Neural Networks on Cardiac Signals From Epicardially Placed Accelerometer. IEEE Journal of Biomedical and Health Informatics 2022; 26:4450-4461. doi:10.1109/JBHI.2022.3181148.
22. Zolgharni M, Negoita M, Dhutia N M, Mielewczik M, Manoharan K, Sohaib S M A, et al. Automatic detection of end-diastolic and end-systolic frames in 2D echocardiography. Echocardiography 2017; 34:956-967. doi:10.1111/echo. 13587.
23. De Caprio L, Ferro G, Cuomo S, Volpe M, Artiaco D, De Luca N, et al. QT/QS2 ratio as an index of autonomic tone changes. Am J Cardiol 1984; 53:818-822. doi:10.1016/0002-9149 (84) 90411-9.
24. Boudoulas H, Geleris P, Lewis R P, Leier C V. Effect of increased adrenergic activity on the relationship between electrical and mechanical systole. Circulation 1981; 64:28-33. doi:10.1161/01.CIR.64.1.28.
25. Paiva R P, Carvalho P, Couceiro R, Henriques J, Antunes M, Quintal I, et al. Beat-to-beat systolic time-interval measurement from heart sounds and ECG. Physiol Meas 2012; 33:177-194. doi:10.1088/0967-3334/33/2/177.
26. Bagal U R, Pandey P C, Naidu S M M, Hardas S P. Detection of opening and closing of the aortic valve using impedance cardiography and its validation by echocardiography. Biomed Phys Eng Express 2017; 4:015012. doi:10.1088/2057-1976/aa8bf5.

27 Pfeiffer E R, Tangney J R, Omens J H, McCulloch A D. Biomechanics of Cardiac Electromechanical Coupling and Mechanoelectric Feedback. J Biomech Eng 2014; 136:0210071-02100711. doi:10.1115/1.4026221.
28. Biering-Sorensen T, Jensen J S, Andersen H U, Rossing P, Jensen M T. Cardiac time intervals and the association with 2D-speckle-tracking, tissue Doppler and conventional echocardiography: the Thousand&1 Study. Int J Cardiovasc Imaging 2016; 32:789-798. doi:10.1007/s10554-016-0839-7.
29. Aase S A, Torp H, Stoylen A. Aortic valve closure: relation to tissue velocities by Doppler and speckle tracking in normal subjects. European Journal of Echocardiography 2008; 9:555-559. doi:10.1093/ejechocard/jen120.
30. Işilay Zeybek Z M, Racca V, Pezzano A, Tavanelli M, Di Rienzo M. Can Seismocardiogram Fiducial Points Be Used for the Routine Estimation of Cardiac Time Intervals in Cardiac Patients? Frontiers in Physiology 2022; 13.
31. Dehkordi P, Khosrow-Khavar F, Di Rienzo M, Inan O T, Schmidt S E, Blaber A P, et al. Comparison of Different Methods for Estimating Cardiac Timings: A Comprehensive Multimodal Echocardiography Investigation. Frontiers in Physiology 2019; 10.

32 Wiens A D, Johnson A, Inan O T. Wearable Sensing of Cardiac Timing Intervals From Cardiogenic Limb Vibration Signals. IEEE Sensors Journal 2017; 17:1463-1470. doi:10.1109/JSEN.2016.2643780.
33. Suh W M, Wong C, Krishnan S C. Importance of electrocardiogram the assessment and management of heart failure: a case report. Journal of Electrocardiology 2008; 41:44-48. doi:10.1016/j.jelectrocard.2007.07.015.
34 Michalski T A, Pszczola J, Lisowska A, Knapp M, Sobkowicz B, Kaminski K, et al. ECG in the clinical and prognostic evaluation of patients with pulmonary arterial hypertension: an underestimated value. Ther Adv Respir Dis 2022; 16:17534666221087846. doi:10.1177/17534666221087846.
35. Gunaseelan R, Sasikumar M, Nithya B, Aswin K, Ezhilkugan G, Anuusha S S, et al. Early/Subtle Electrocardiogramatures of Acute Coronary Syndrome and S T-Segment Elevation Myocardial Infarction. J Emerg Trauma Shock 2022; 15:66-69. doi:10.4103/JETS.JETS_186_20.
36. Lang R M, Badano L P, Mor-Avi V, Afilalo J, Armstrong A, Ernande L, et al. Recommendations for Cardiac Chamber Quantification by Echocardiography in Adults: An Update from the American Society of Echocardiography and the European Association of Cardiovascular Imaging. European Heart Journal-Cardiovascular Imaging 2015; 16:233-271. doi:10.1093/ehjci/jev014.
37. Kashou A H, Mulpuru S K, Deshmukh A J, Ko W-Y, Attia Z I, Carter R E, et al. An artificial intelligence-enabled ECG algorithm for comprehensive ECG interpretation: Can it pass the ‘Turing test’? Cardiovascular Digital Health Journal 2021; 2:164-170. doi:10.1016/j.cvdhj.2021.04.002.
38. Vaid A, Johnson K W, Badgeley M A, Somani S S, Bicak M, Landi I, et al. Using Deep-Learning Algorithms to Simultaneously Identify Right and Left Ventricular Dysfunction From the Electrocardiogram. JACC: Cardiovascular Imaging 2022; 15:395-410. doi:10.1016/j.jcmg.2021.08.004.
39. Schlesinger D E, Diamant N, Raghu A, Reinertsen E, Young K, Batra P, et al. A Deep Learning Model for Inferring Elevated Pulmonary Capillary Wedge Pressures From the 12-Lead Electrocardiogram. JACC: Advances 2022; 1:1-11. doi:10.1016/j.jacadv.2022.100003.
40. Gill H, Hoffmann A. The timing of onset of mechanical systole and diastole in reference to the QRST complex: a study to determine performance criteria for a non-invasive diastolic timed vibration massage system in treatment of potentially unstable cardiac disorders. Cardiovasc Eng 2010; 10:235-245. doi:10.1007/s10558-010-9108-x.
41 Obata Y, Ruzankin P, Gottschalk A, Berkowitz D E, Steppan J, Barodka V. The Effect of Exercise on the Timing of Aortic Valve Closure with Respect to the ECG Tracing. International Journal of Cardiovascular Research 2017; 2017. doi:10.4172/2324-8602.1000318.
42. Vazquez-Seisdedos C R, Neto J E, Maranon Reyes E J, Klautau A, Limao de Oliveira R C. New approach for T-wave end detection on electrocardiogram: Performance in noisy conditions. BioMedical Engineering OnLine 2011; 10:77. doi:10.1186/1475-925X-10-77.
43 Laguna P, Thakor N V, Caminal P, Jane R, Yoon H-R, Bayes de Luna A, et al. New algorithm for Q T interval analysis in 24-hour Holter ECG: performance and applications. Med Biol Eng Comput 1990; 28:67-73. doi:10.1007/BF02441680.
44 Little R C. The mechanism of closure of the mitral valve: a continuing controversy. Circulation 1979; 59:615-618. doi:10.1161/01.CIR.59.4.615.
45. Pellerin D, Larrazet F, Cohen L, Witchitz S, Veyrat C. Myocardial time intervals preceding left ventricular filling in chronic coronary artery disease: value of a decreased septal ejection time. Int J Cardiol 2003; 89:33-44. doi:10.1016/s0167-5273 (02) 00422-9.
46. Vlasseros I, Katsi V, Vyssoulis G, Pylarinos I, Ioannis P, Richter D, et al. Aggravation of left ventricular diastolic dysfunction in hypertensives with coronary artery disease. Hypertens Res 2013; 36:885-888. doi:10.1038/hr.2013.67.

1. A method for determining cardiac events comprising:
obtaining, by a computing device, a physiological data signal of a heart of an individual;
identifying, by the computing device, features of the physiological data signal and applying the features as inputs to a prediction model;
determining, by the computing device using the prediction model, the cardiac events for one or more valves of the heart, wherein the cardiac events include timing of opening and closing of the one or more valves of the heart; and
outputting, by the computing device, the cardiac events determined using the prediction model.

2. The method of claim 1, wherein the one or more valves of the heart comprise the aortic valve and the mitral valve.

3. The method of claim 2, wherein the physiological data signal comprises a left ventricular pressure data signal.

4. The method of claim 3, wherein the features comprise first and second derivatives of the left ventricular pressure data signal.

5. The method of claim 1, further comprising synchronizing timing of the physiological data signal with another modality measurement of the heart using the timing of the opening and closing of the one or more valves of the heart.

6. The method of claim 5, wherein the physiological data signal comprises a left ventricular pressure data signal and the another modality measurement comprises a left ventricular volume data recording of the heart that is acquired non-simultaneously with the left ventricular pressure data signal.

7. The method of claim 6, further comprising generating, by the computing device, a synchronized pressure-volume loop display by aligning the non-simultaneously acquired left ventricular volume data with the non-simultaneously acquired left ventricular pressure data.

8. The method of claim 1, wherein the physiological data signal comprises an electrocardiogram signal.

9. The method of claim 8, wherein the features comprise R wave, S wave, and end of T wave features of the electrocardiogram signal.

10. The method of claim 9, wherein the R wave, S wave, and T waves are identified by analyzing maxima and minima of the electrocardiogram signal, wherein the end of T wave is identified using a second order derivative of the electrocardiogram signal.

11. A system for determining cardiac events comprising:
a processor of a computing device; and
a memory in communication with the processor, the memory storing program instructions, the processor operative with the program instructions to perform the operations of:
obtaining a physiological data signal of a heart of an individual;
identifying features of the physiological data signal and applying the features as inputs to a prediction model;
determining, using the prediction model, the cardiac events for one or more valves of the heart, wherein the cardiac events include timing of opening and closing of the one or more valves of the heart; and
outputting the cardiac events determined using the prediction model.

12. The system of claim 11, wherein the one or more valves of the heart comprise the aortic valve and the mitral valve.

13. The system of claim 12, wherein the physiological data signal comprises a left ventricular pressure data signal.

14. The system of claim 13, wherein the features comprise first and second derivatives of the left ventricular pressure data signal.

15. The system of claim 11, wherein the operations further comprise synchronizing timing of the physiological data signal with another modality measurement of the heart using the timing of the opening and closing of the one or more valves of the heart.

16. The system of claim 15, wherein the physiological data signal comprises a left ventricular pressure data signal and the another modality measurement comprises a left ventricular volume data recording of the heart that is acquired non-simultaneously with the left ventricular pressure data signal.

17. The system of claim 16, wherein the operations further comprise generating a synchronized pressure-volume loop display by aligning the non-simultaneously acquired left ventricular volume data with the non-simultaneously acquired left ventricular pressure data.

18. The system of claim 11, wherein the physiological data signal comprises an electrocardiogram signal.

19. The system of claim 18, wherein the features comprise R wave, S wave, and end of T wave features of the electrocardiogram signal.

20. The system of claim 19, wherein the R wave, S wave, and T waves are identified by analyzing maxima and minima of the electrocardiogram signal, wherein the end of T wave is identified using a second order derivative of the electrocardiogram signal.
